(19) United States
(12) Patent Application Publication
(10) Pub. No.: US 2019/0362430 A1
(43) Pub. Date: Nov. 28, 2019

(54) Electronic fulfillment system and method for completing life insurance settlement transactions and obtaining and managing electronic signatures for life insurance settlement transaction documents

(71) Applicant: Stephen Jass, Deerfield Beach, FL (US)

(72) Inventor: Stephen Jass, Deerfield Beach, FL (US)

(21) Appl. No.: 16/391,427

(22) Filed: Apr. 23, 2019

Related U.S. Application Data
(60) Provisional application No. 62/661,172, filed on Apr. 23, 2018.

(51) Int. Cl. G06Q 40/08; G16H 10/60

(52) U.S. Cl. CPC G06Q 40/08; G16H 10/60

(57) ABSTRACT

A partially-automated life insurance settlement system and method is provided for processing and preparing life insurance settlement transaction documents, back-office requirement order management, user hierarchy network management, funding source presentation, offer negotiation and life insurance settlement transaction management. A data management device is interconnected with a plurality of terminals to permit data to be entered and retrieved from the data management device. The data management device is also provided with the capability of merging entered or stored data with life insurance settlement transaction documents. These documents are then transmitted for electronic signature or to be printed, signed, digitized and uploaded. Signed documents are then processed by a back-office to gather additional documents. Funder source presentation, distribution, inventory and offer management capabilities then manage pricing and offer steps of a transaction.

### CROSS-REFERENCE TO RELATED APPLICATIONS

This application claims priority to U.S. Provisional Patent Application No. 62/661,172 filed on Apr. 23, 2018 by Stephen Jass entitled “Electronic fulfillment system and method for completing life insurance settlement transactions and obtaining and managing electronic signatures for life insurance settlement transaction documents,” the entire contents of which are incorporated by reference herein.

### TECHNICAL FIELD

The present invention relates to the steps involved in a life insurance settlement transaction, more particularly, to obtaining signatures on documents related to a life insurance settlement transaction, more particularly, to the use of signed life insurance settlement transaction documents to process a life insurance settlement transaction, more particularly, to using a blockchain to prove the validity of original life insurance settlement transaction documents, more particularly, to the use of user hierarchy networks to encourage industry expansion, and, more particularly, to processing life insurance settlement transactions in order to provide policy owners offers for their existing life insurance. Specifically, the present invention relates to a system and method of improving the transaction of life insurance settlement to decrease transaction time, simplify the process, and, support user hierarchy networks.

### BACKGROUND

The vast majority of life insurance settlement cases are obtained by brokers, agents, financial professionals or providers (“referral parties”) who often represent these obtained cases to a plurality of funding sources. These referral parties must track the completion of life insurance settlement application documents, the ordering of all required documents (e.g., medical records, illustrations, policy values and life expectancies), the packaging of all life insurance settlement documents to be sent to funding sources, sending documents to funding sources, managing funding source interest, presenting any offers to policy owners, negotiating offers, and, if an offer is accepted, managing the case through the closing process. To complicate matters, the application forms that must be completed by potential policy owners to begin a life insurance settlement transaction may vary by state. Because most referral parties may only submit a few life insurance settlement transactions each year they lack the repetition and familiarity necessary to fully understand how to properly complete life insurance settlement applications. This confusion may lead to data entry and processing errors, which result in costly transaction delays and lost sales opportunities. Because the life insurance settlement application is the trigger mechanism that initiates the ordering of requirements (e.g., medical records, carrier illustrations, medical summaries and life expectancy reports) and case distribution processes, it is critical that the application be completed correctly. Furthermore, these ordering of requirements processes often strain available resources which results in delays due to lack of follow-up. Furthermore, referral parties may belong to user hierarchy networks with users at the same level, above and/or bellow them (e.g., a financial professional user submitting a case is under an agent user who is under a general agent user who is under a broker user). It becomes a strain to manage user hierarchy network case submission and has been a reason why some user hierarchy networks have not participated in the life settlement industry.

There has never been a solution developed to address the fulfillment of life insurance settlement applications. Furthermore, there has never been a solution developed to address the ordering requirements processes, user hierarchy networks, and offer & closing management in one system.

Published U.S. Pat. No. 8,285,569 B2 to Spalding, Jr. claims a system for facilitating life settlement transactions. Unfortunately, these claims focus on cases being presented to funding sources and does not focus on case submission, transaction document management or user hierarchy networks. This patent was also intended to eliminate the current way the life insurance settlement industry manages offers. Currently referral parties receive offers, revise those offers and make revised offers to the next referral party down until a final revised offer reaches the policy owner. Furthermore, by focusing primarily on funding sources these claims have failed to address the actual frustrations and obstacles encountered by parties to life insurance settlement transactions every day regarding submission issues such as application completion, requirement ordering methods & user hierarchy network expansion.

Currently, documents for life insurance settlement transactions (e.g., application, authorizations, purchase and sale agreements, escrow agreements, closing contracts, life insurance carrier forms, etc.) are created by the various parties involved in the transaction, such as life insurance settlement brokers, providers, agents, attorneys, etc. During the transaction, documents are printed and presented to the parties to the transaction (e.g., policy owners, insureds and funding sources) for signature. However, often the parties do not review the documents and/or do not sign the documents in all the required positions for signatures, which thus delays completion of the transaction. Furthermore, particular insurance carriers & medical offices require unique authorizations to be signed in order to release documents used to make offers on life insurance settlement cases. Delays in completion of a transaction may result in extra costs to the purchaser or policy owner (e.g., payment of additional premiums), and to all parties due to time lost and absolute loss if a life insurance policy lapses during the transaction process.

Therefore, it would be desirable to provide a system and method which is able to initiate life insurance settlement transactions, allow for electronic signature of life insurance settlement documents, manage the ordering requirements processes, distribute cases to funding sources, track funding source interest, provide a means for all parties to negotiate an offer, and manage the closing of a life insurance settlement transaction if an offer is agreed upon. This life insurance settlement transaction focused approach addresses application, processing and funding source management frustrations. Furthermore, it would be desirable to provide a system and method which is able to allow the creation of user hierarchy networks so that organizations with many users can manage life insurance settlement in a structured and transparent way across an entire organization.

### BRIEF SUMMARY OF THE INVENTION

The present invention provides a partially automated life insurance settlement transaction fulfillment system and method. The system and method provides a process through web based forms and allows for generated state specific life insurance application documents to be either electronically signed or printed, signed, digitized & uploaded. The invention also provides a system and method for gathering additional life insurance settlement documents needed for pricing (e.g., medical records, illustrations and life expectancy reports). The invention also provides a system and method for distributing assembled cases to funding sources to price and make offers. The invention also provides a system and method for managing offers from funding sources. The invention also provides a system and method for managing the closing process allowing for closing documents to be either printed, signed, notarized, digitized & uploaded or electronically signed and notarized. The invention also provides a system and method for generating blockchain “digital fingerprint” for every document for future authenticity verification. The invention also provides a system and method for the creation of user hierarchy networks.

The present invention specifies systems and methods for life insurance settlement transactions which meets the goals of reducing broker data processing errors, increasing broker productivity, decreasing transaction time, and expanding industry involvement through user hierarchy networks.

The details of one or more embodiments of the invention are set forth in the accompanying drawings and the description below. Other features, objects, and advantages of the invention will be apparent from the description and drawings, and from the claims.

### BRIEF DESCRIPTION OF DRAWINGS

FIG. 1 illustrates an example of a system for obtaining electronic signatures and generating a digital fingerprint of signed documents on a blockchain.

FIG. 2A-B illustrates an example of a system and method for obtaining and managing life insurance settlement transaction documents.

FIG. 3A-C illustrates an example of a system and method for managing life insurance settlement transactions.

FIG. 5 illustrates an example of a hardware diagram of components of the system for managing life insurance settlement transactions.

FIG. 6A-E provide a running flow diagram of one embodiment of the system and method.

FIG. 7 illustrates an example of a life insurance settlement application form screen.

FIG. 8 illustrates an example of a life insurance settlement document signature options screen.

FIG. 9 illustrates an example of a life insurance settlement status screen.

FIG. 10 illustrates an example of a user hierarchy network.

FIG. 11 illustrates an example of a user information screen.

### DETAILED DESCRIPTION OF THE INVENTION

The following detailed description, given by way of method and system examples, are not intended to limit the present invention solely to the described embodiments.

Life Insurance Settlement transactions (e.g., application, secondary settlement, tertiary settlement, etc.) may involve a variety of documents, (e.g., disclosures, authorizations, releases, carrier change forms, etc.). Systems and processes may obtain and manage (e.g., capture, digitize, store, secure, apply and blockchain verify) signatures of life insurance settlement transaction documents. Life insurance settlement transaction documents may be presented to users for review and/or approval. Positions in the documents (e.g., areas or portions of a document) that require signatures may be identified and presented for electronic signature. Users may electronically sign positions in documents, as appropriate. These signed documents may be used to complete requirement ordering processes, case distribution, offer management and the closing process. Systems and processes may allow the creation of user hierarchy networks.

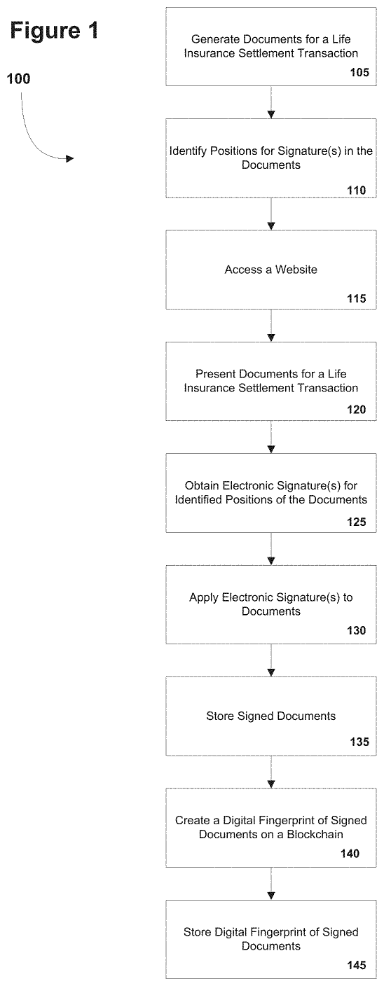

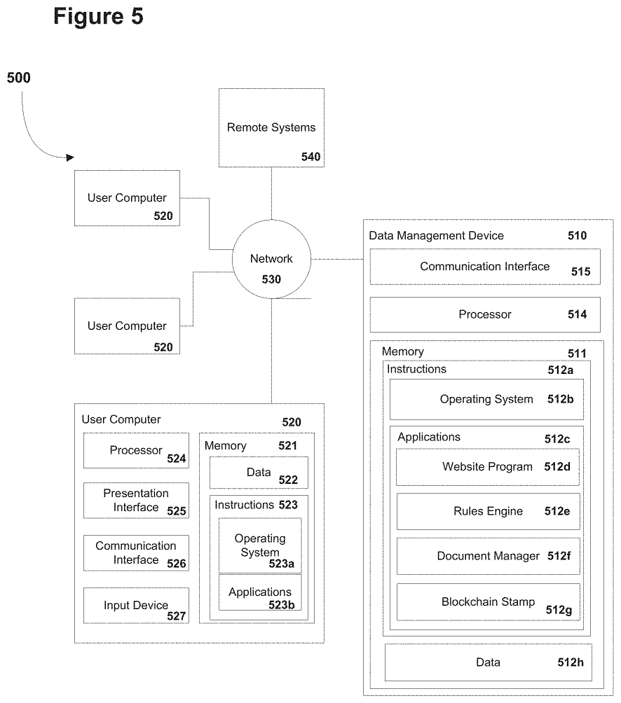

FIG. 1 illustrates a process for obtaining and managing signatures for life insurance settlement transaction documents that may be performed by systems, such as system 500 illustrated in FIG. 5. Documents for the life insurance settlement transaction may be generated (operation 105). For example, a centralized repository may include information provided by one or more parties to a life insurance settlement transaction and the documents related to the life insurance settlement transaction may be generated based on the information stored in the repository. Documents, such as life settlement applications, authorization documents, closing documents, life insurance carrier documents and other documents, may also be retrieved or otherwise accessed from a memory, for example, for an application for the sale of a life insurance policy through a life insurance settlement.

Positions for signature in the documents may be identified (operation 110). For example, signature indicators in documents may be identified. Signature indicators in documents may include metadata, such as text (e.g., policy owner name, party type, etc.), and the system may identify the text and present the signature block associated with the text for signature by the user, as appropriate. The text may be white or the color of the background of the document so that this text does not appear when the document is printed. As another example, a signature indicator may include metadata or other software key that identifies the signature block. The system may identify the metadata or software key and present the portion associated with the metadata for signature by a user. The metadata or software key may identify the person whose signature is required in the signature block (e.g., insured, policy owner, etc. should sign the signature block).

A website may be accessed (operation 115) by a user. For example, a party to a life insurance settlement transaction (e.g., primary insured, secondary insured, agent, policy owner, funding source, etc.) may access a website coupled to a data management system.

The documents for the life insurance settlement transaction may then be presented (operation 120). For example, a user may be presented with the document or portions thereof for review and/or approval on the website. A user may be required (e.g., by government and/or industry regulations) to indicate approval and/or the ability to view the documents prior to being able to electronically sign the documents.

Electronic signatures for the identified positions may then be obtained (operation 125). For example, a user may sign a digital signature pad coupled to a user computer. As another example, an indication of the user's signature may be used to sign the identified positions (e.g., indicating signature by typing the user's name or selecting a button that indicates that a signature should be added to the document). The positions for signature may be presented automatically to a user (e.g., sequentially) and the user may sign the documents or portions thereof (e.g., using a digital signature pad or other type of electronic signature) or not sign the documents or portions thereof (e.g., when reviewing the document as opposed to signing the document). Signature may be required prior to proceeding to sign other portions of the document, in some implementations. This may reduce the number of missing signatures from the documents. Reducing missed signatures may reduce delays to parties to the transaction that might be caused by the missing signatures. In addition, reducing the risk of missing signatures may reduce costs to parties that facilitate the transactions, such as a provider company, since a thorough a review of the signatures may not be required (e.g., when signature is required before proceeding to later signatures, when a signature sequence of the signature blocks is specified and signing out of order is inhibited).

In some implementations, rather than applying the signature to each position, the signature may be applied to more than one position or the entire document concurrently. The system may apply the user signature to the entire document. (operation 130). The signed documents may be stored (operation 135). For example, the signed documents may be stored in a memory of the system and/or on remote systems, such as a life insurance carrier memory system.

The signed documents may have a digital fingerprint created (operation 140), containing a blockchain timestamp and proof document. The digital fingerprint may be stored (operation 145). For example, the signed documents may be sent by an API to a blockchain digital fingerprint generation system (e.g., such as blockchain timestamping commercially available from Stampery, Inc., San Francisco, Calif.) and the proof document returned to be stored in a memory of the system and/or on remote systems.

Process 100 may be implemented by system 500 or similar systems. In addition, various operations may be added, deleted, modified, or reordered in process 100. For example, positions in the document may be identified based on the user logged onto the system. When a policy owner is logged onto the system, the documents and/or positions in the documents for signature by the policy owner may be identified and presented to the policy owner. However, when an agent is logged onto the system, similar and/or different documents or signature positions may be identified and/or presented to the agent user. As another example, documents may be generated by the system prior to retrieving the documents. In addition, electronic signatures may be added to the document as they are received for a document or after signature of all signature blocks of the document. As another example, after signature by all parties, a secure version of the signed documents may be saved and/or transmitted to remote systems. Secure versions may identify the date of creation and/or signature. Secure versions may also inhibit alteration of documents. In addition, the signed documents may be automatically transmitted (e.g., to back-office service providers, to brokers, to buyers, to policy owners, etc.). Furthermore, signatures on the electronic documents may be verified (e.g., using forensic information related to signatures and/or biometric information and/or blockchain digital fingerprint).

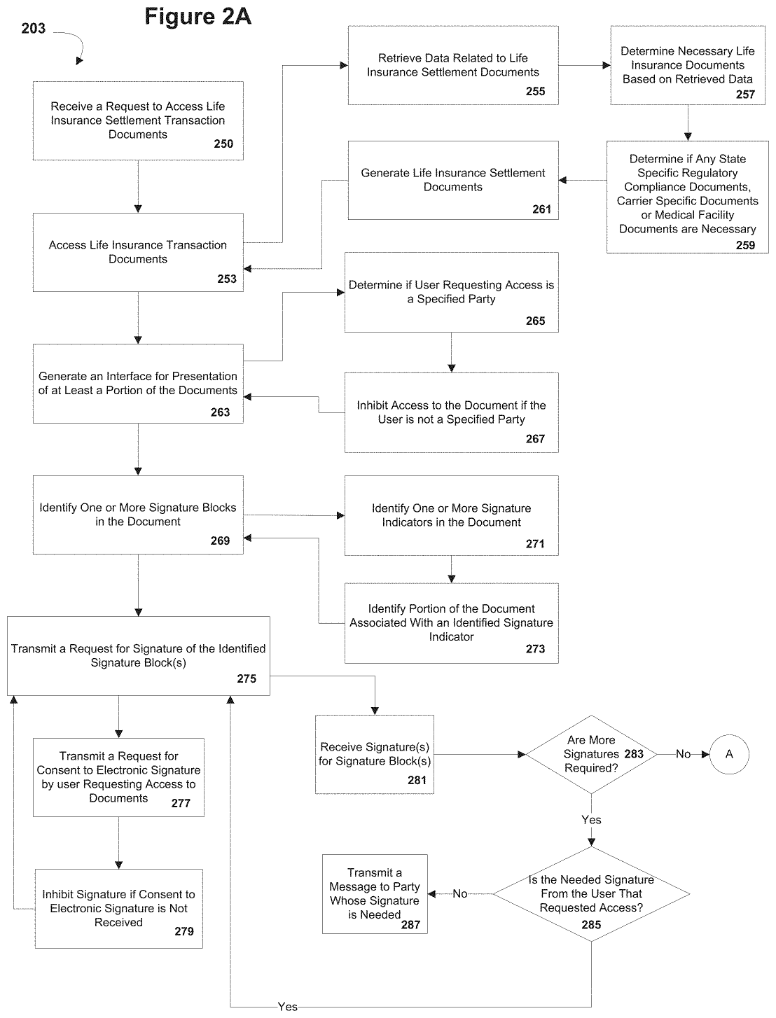

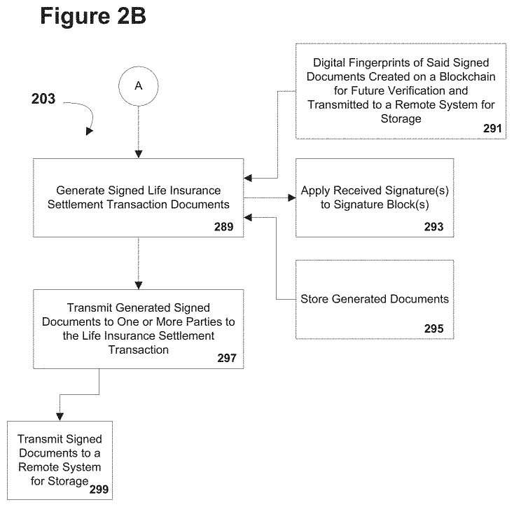

FIGS. 2A-B illustrates an example of a system and method for obtaining and managing life insurance settlement transaction documents. A request to access life insurance settlement transaction documents may be received (operation 250). For example, a user may access a website on the Internet to request access to documents related to the sale of a life settlement policy.

Life insurance settlement transaction documents may be accessed (operation 253). Data related to the life insurance settlement transaction documents may be retrieved (operation 255), a rules engine may determine the necessary transaction documents needed depending on retrieved data (e.g., if policy owner is not the insured, if the policy owner declared bankruptcy, etc.) (operation 257), a rules engine may determine if any state specific regulatory compliance documents (e.g., state disclosures, state life settlement information documents, etc.), life insurance carrier specific documents (e.g., third party authorizations, illustration request forms, etc.), and, medical facility custom release forms are necessary (operation 259), and the life insurance settlement documents may be generated (operation 261). For example, data specific to the life insurance settlement transaction may be retrieved and the data may be used to populate forms (e.g., stored in a memory of the system) for use in the life insurance settlement transaction.

An interface for presentation of at least a portion of the documents may be generated (operation 263). The interface may facilitate viewing and reviewing of the life insurance settlement transaction documents by the user requesting access. A determination may be made whether the user requesting access is a specified party (operation 265) and access to the life insurance settlement transaction documents may be inhibited if the user is not a specified party (operation 267). For example, access to life insurance settlement transaction documents may be restricted. In some implementations, specified parties, such as parties to the transaction (e.g., policy owner, insured, etc.) and parties facilitating the transaction (e.g., agent, broker, provider, etc.), may be allowed to access the documents, while others are inhibited from accessing the documents. Specified parties may be identified by user information provided by a user. The type of access allowed by each party may also depend on the user and their place within a user hierarchy network. For example, policy owners may be inhibited from modifying information on the life insurance settlement transaction documents (e.g., carrier name, policy face amount, etc.); agents may be allowed to modify a portion of the information (e.g., insured name, policy owner name, etc.), but may be inhibited from modifying other types of information (e.g., Social Security Number, Date of Birth, etc.).

One or more signature blocks in the document may be identified (operation 269). For example, signature blocks for a policy owner may be identified. In some implementations, the signature blocks may be identified at least partially based on the user. As an example, if the user requesting access is the policy owner, the signature blocks for policy owner may be identified and the signature blocks for the agent may not be identified. Signature indicator(s) in the document may be identified to identify the signature blocks in the document (operation 271). For example, signature indicators may include metadata in life insurance settlement transaction documents. A search may be performed to identify the metadata in the life insurance settlement transaction documents that are signature indicators. As another example, a signature indicator may be text that is white (e.g., such that the text may be identified by the system, but may not be presented or viewable by a user) or hidden. A search for the white text or hidden text may be performed to identify the signature blocks in life insurance settlement transaction documents. As another example, software keys may mark positions of the signature blocks in a life insurance settlement transaction documents.

The portion of the document associated with the identified signature indicator may also be identified (operation 273). The portion of the document may be presented to a user.

A request for signature of the identified signature block(s) may be transmitted (operation 275). For example, a portion of the life insurance settlement transaction document may be presented to the user through the interface and the user may be prompted, through the interface, to sign a signature block associated with the presented portion. A request for consent to electronic signature of the life insurance settlement transaction documents may be transmitted (operation 277). For example, an interface such as a webpage or a pop-up window may be presented to the user with a consent agreement for the use of an electronic signature rather than a handwritten signature. A user may select a link or a button to indicate acceptance. In some implementations, a user may be required to provide a code that appears on the consent agreement to indicate that the user consents to the agreement. Signature of the life insurance settlement transaction documents may be inhibited if the consent to electronic signature is not received (operation 279). For example, the user may not be able to proceed to the interface configured to receive the signature, if the user does not consent to electronic signature of the life insurance settlement transaction documents. As another example, a message may be transmitted to one or more parties (e.g., policy owner, insured, agent, etc.) that the user does not consent to electronic signature. A user may be able to view the life insurance settlement transaction documents even if the consent to electronic signature is not received.

Signature(s) may be received for the signature block(s) (operation 281). For example, a user may utilize a digital signature pad to send a signature for a signature block. As another example, a user may enter his name in an acceptable electronic signature format.

A determination may be made whether more signatures are required (operation 283). For example, a determination may be made whether signatures have not been received for the user requesting access and/or for any users. A determination may be made whether signatures are needed from the user that requested access (operation 285) and if more signatures are required, then a request may be transmitted for signature of the signature blocks that have not been signed (operation 275). If signatures from the user who requested access are not needed, but signatures from other users are required, a message may be transmitted to the party whose signature is needed (operation 287).

Signed life insurance settlement transaction documents may be generated (operation 289). The documents may be generated such that access is restricted (e.g., encrypted) or inhibited. The documents may be generated such that when a document is accessed, access information (e.g., assessor name, time and date of access, type of access such as modification or presentation, etc.) may be obtained. The access information may be associated with and/or stored with the generated documents.

Received signatures may be applied to the signature block(s) of the life insurance settlement transaction documents (operation 293). The generated signed life insurance settlement transaction documents may be stored (operation 295). The generated signed life insurance settlement transaction documents may have a blockchain digital fingerprint generated and stored (operation 291).

The signed life insurance settlement transaction documents may be transmitted to one or more parties to the life insurance settlement transaction (operation 297). For example, the life insurance settlement transaction documents may be emailed to the insured(s), policy owner(s), and/or agent(s). As another example, the life insurance settlement transaction documents may be transmitted to a back-office for processing.

The signed life insurance settlement transaction documents may be transmitted to a remote system for storage (operation 299). The life insurance settlement transaction documents may be securely stored. For example, access may be inhibited. As another example, the life insurance settlement transaction documents may be transmitted to a life insurance carrier system or similar system.

Method 203 may be implemented by system 500 or similar systems. In addition, various operations may be added, deleted, modified, or reordered in process 203. In some implementations, signature indicators may be generated. For example, specified text may be identified (e.g., “policy owner”, “insured”, hidden text, etc.) and the signature indicator may be generated. As another example, when documents are generated, the signature indicator may be generated as well. In some implementations, the user may provide a signature one time and apply the signature to more than one signature block (e.g., provide one signature for the life insurance settlement transaction documents and/or provide one signature for a portion of the life insurance settlement transaction documents). In some implementations, signed documents may be generated even when additional signatures are needed. For example, the signed documents may be generated after the policy owner and/or agent have provided signatures and then generated again after the insured has provided signatures.

In some implementations, a notary public (“notary”) may witness signatures for life insurance settlement transaction documents. A notary may be inhibited from signing life insurance settlement transaction documents until a user, whose signature is being witnessed, has signed the life insurance settlement transaction documents. A notary may witness and/or provide a signature indicating the witnessing of a signature for each signature block or for the life insurance settlement transaction documents.

In some implementations, a sequence for the signature of documents may be specified. For example, signature of other portions of the life insurance settlement transaction documents (e.g., closing documents) may be inhibited until an offer acceptance for the life insurance settlement transaction documents is signed. As another example, signature of a consent (e.g., consent to electronic signature or other appropriate consents) may be requested prior to allowing signature of the life insurance settlement transaction documents. Signature of the life insurance settlement transaction documents may be inhibited unless the signature of the consent is received. In some implementations, the sequence for the signature of the documents may be retrieved (e.g., from a memory of the system), portions of the documents may be presented through the interface in the sequence for signature, and/or signatures may be requested based on the specified sequence. Signature of the life insurance settlement transaction documents may be inhibited if the signatures are not provided according to the specified sequence.

In some implementations, the signed life insurance settlement transaction documents may be generated after the parties have transmitted electronic signatures for the life insurance settlement transaction documents. Each party may transmit signatures for the life insurance settlement transaction documents separately and/or concurrently.

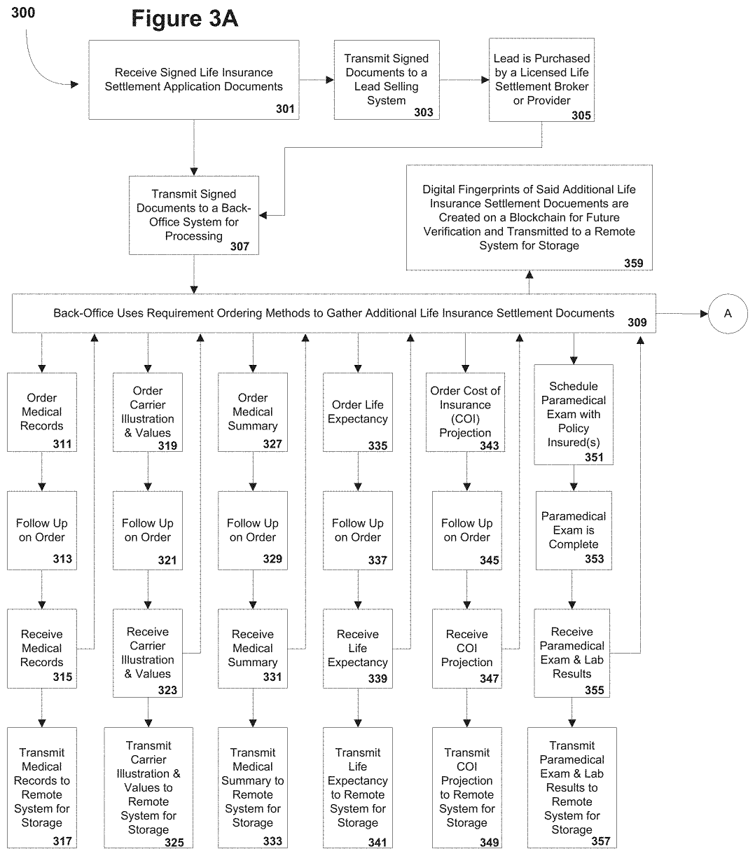

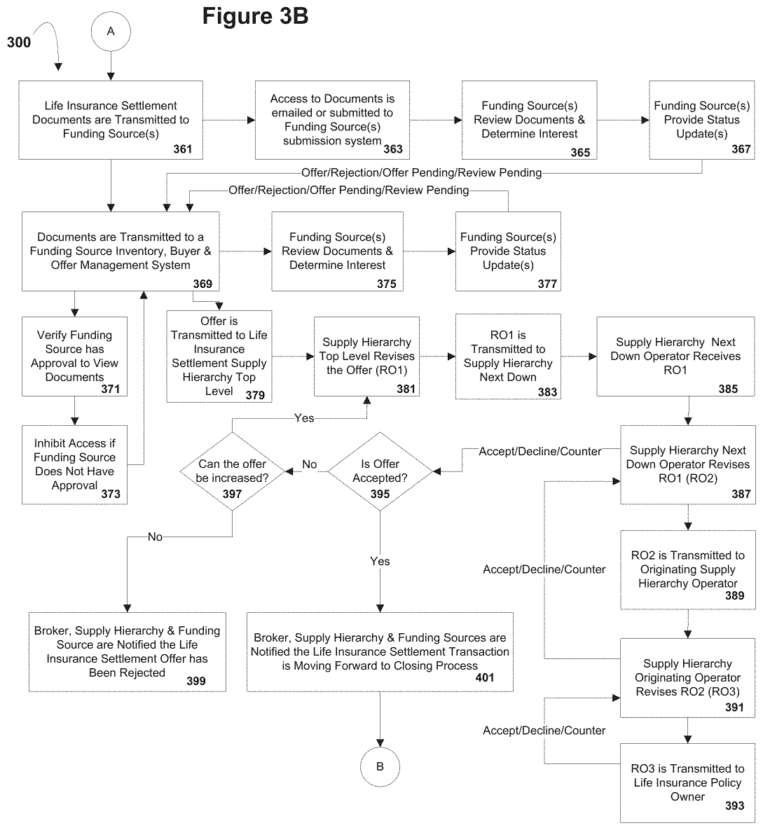

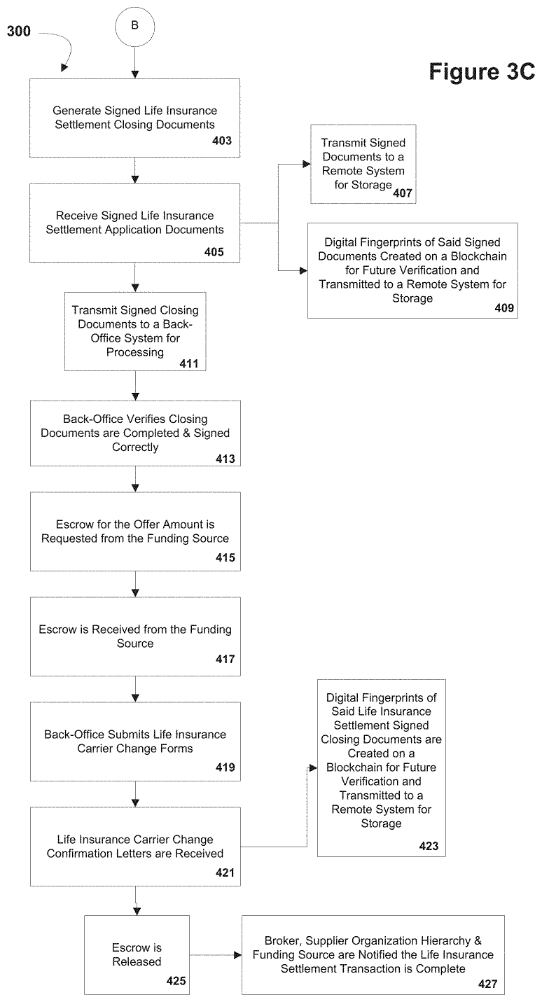

FIGS. 3A-C illustrates an example of a system and method for managing life insurance settlement transactions. Signed life insurance settlement application documents may be received (operation 301). For example, a user may access a website on the Internet to transmit signed life insurance settlement application documents to a system. As another example, a user may electronically sign life insurance settlement application documents through method 203 and these signed documents be used in system and method 300.

Signed life insurance settlement application documents may be sold as leads (operation 303) to a licensed life settlement broker or provider (operation 305). For example, a received signed life insurance settlement application could be sold through an auction between several brokers and providers.

Signed life insurance settlement application documents may be transmitted to a back-office system for processing (operation 307). The back-office system may use requirement ordering methods to gather additional life insurance settlement documents (operation 309). For example, the back-office may order medical records (operation 311), follow up on the medical record order(s) (operation 313), receive medical records (operation 315) and, transmit medical records to the back office as well as to a remote system for storage (operation 317). As another example, the back-office may apply these requirement ordering methods to gather other requirements needed for funding sources to make offers on a life insurance settlement case (e.g., carrier illustrations & values (operations 319-325), medical summaries (operations 327-333), life expectancy reports (operations 335-341), cost of insurance projections (operations 343-349), paramedical exams (operations 351-357), etc.). The gathered additional life insurance settlement requirement documents may have a blockchain digital fingerprint generated and stored (operation 359). Funding sources may receive access to all life insurance settlement documents or only have access to some of the life insurance settlement documents.

Life insurance settlement documents may be transmitted to funding source(s) (operation 361). For example, a funding source may be sent access to life insurance settlement documents through an email (operation 363). As another example, life insurance settlement documents may be transmitted to a website inventory system that will present available life insurance settlement cases and give access to each case life insurance settlement documents (operation 369). A funding source may have access to all life insurance settlement documents or may need to be verified as having approval to view documents (operation 371). A funding source may not have approval and be inhibited from viewing some or all documents (operation 373). Some documents may have certain identifying information redacted (e.g., policy owner phone number, agent name, etc.). Funding Sources may receive access to life insurance settlement documents through both email and website presentation system.

Funding sources may review life insurance settlement documents to determine interest (operation 363 & 375) and provide status updates (e.g., review pending, offer pending, offer, rejection, etc.) (operation 367 & 377). For example, a funding source may review medical records and policy illustration(s) and determine they have no interest in a particular life insurance settlement case. As another example, a funding source may review medical records and policy illustration(s) and determine they would like to make a cash plus retained death benefit offer for the purchase of a particular life insurance settlement case.

Funding sources may make offers (operations 367 & 377) on a particular life insurance settlement policy. These offers may be transmitted to the life insurance settlement supply user hierarchy network top level (e.g., life insurance settlement broker, provider, policy owner, etc.) (operation 379). The supply user hierarchy network top level may revise this offer (operation 381). The supply hierarchy top level may transmit may transmit this revised offer to the Supply Hierarchy Next Down (operation 383). The supply hierarchy next down may receive this revised offer (operation 385). The supply hierarchy next down may revise this offer (operation 387) and transmit the second revised offer to the supply hierarchy originating supplier (operation 389). The supply hierarchy originating supplier may revise this offer (operation 391) and transmit the third revised offer to the life insurance policy owner (operation 393). The supply hierarchy may contain many or no levels in between the funding source and life insurance policy owner. For example, a funding source may transmit an offer of $100,000; the supply hierarchy top level may revise this offer to $95,000 and transmit this first revised offer to the supply hierarchy next down; the supply hierarchy next down may revise this offer from $95,000 to $90,000 and transmit this second revised offer to the supply hierarchy originating supplier; the supply hierarchy originating supplier may revise this offer from $90,000 to $80,000; this third revised offer can then be transmitted to the life insurance policy owner.

In some implementations, some or all members of the supply user hierarchy network will receive a flat fee or percentage of an offer. Therefore, they will not necessarily need to review each offer nor actively be involved in the offer process.

The life insurance policy owner may provide feedback on the offer they are presented (e.g., accept, decline, counter, etc.). This feedback is provided back up the supply user hierarchy network and the life insurance policy owner may accept the offer (operation 395). If the offer is not accepted, and the offer can be increased, the supply user hierarchy network can revise the offer again and the bid-revise-bid method can continue until the life insurance policy owner accepts an offer. The supply user hierarchy network top level may need to go back to the funding source and provide a counter offer to them in order to increase the funding source offer. If the offer can not be increased the life insurance settlement parties (e.g., broker, supply hierarchy, funding source(s)) may be notified the offer has been rejected (operation 399). If the offer is accepted the life insurance settlement parties (e.g., broker, supply hierarchy, funding source(s), etc.) may be notified the life insurance settlement transaction is moving forward to closing (operation 401).

Once an offer is accepted and a life insurance settlement is moving forward to closing, the method and system may generate signed life insurance settlement closing documents (operation 403). The generation of life insurance settlement closing documents may be implemented by the example system and method 203. For example, all life insurance settlement parties (e.g., policy owner, agent, insured, funding source, etc.) may receive a request for information needed to generate life insurance settlement closing documents and once documents are generated be notified of any portions of the life insurance settlement closing documents they need to sign with an option sign the documents using electronic signature.

The signed life insurance settlement closing documents (e.g., disclosures, authorizations, life insurance carrier transfer & conversion forms, etc.) may be received (operation 405), transmitted to a remote system for storage (operation 407) and, have a blockchain digital fingerprint of signed documents created (operation 409).

The signed life insurance settlement closing documents may be transmitted to a back-office for processing (operation 411). The back-office may use processing methods to verify the closing documents are completed & signed correctly (operation 413). For example, the back-office may identify a required closing document that is missing and request said missing document from the life insurance settlement parties.

Once closing documents are received in good order, escrow for the offer amount may be requested from the funding source (operation 415). Once the escrow has been verified as received (operation 417) the back-office obtains signatures from the funding source on carrier change forms and submits the signed life insurance carrier change forms (operation 419). The life insurance carrier changes are tracked and change of ownership & change of beneficiary confirmation letters are received (operation 421). The carrier confirmation letters may have a blockchain digital fingerprint generated (operation 423). Once the carrier confirmation letters are received the escrow may be released (operation 425). With the life insurance settlement transaction complete the life insurance settlement parties (e.g., policy owner, insured, funding source, etc.) may be notified the transaction is complete (operation 427).

FIG. 5 illustrates an example system 500 for obtaining and managing storage & signatures for life insurance settlement transaction documents as well as user hierarchy network management. An enterprise (e.g., corporation, service provider, etc.) may have a data management device to facilitate management of life insurance settlement data and user hierarchy network(s). Life insurance settlement transactions often include data and numerous documents. Frequently, additional documents will be added & changes may be made to the documents after initial creation of a document and, thus, including a centralized repository through which the many parties of a life insurance settlement transaction may interact on a controlled and secure basis, may improve life insurance settlement transaction management.

A data management device 510 may be coupled to one or more user computers 520 via a network 530. The data management device 510 may be a server or other computer system that includes a memory 511. Instructions 512a, operating systems 512b, and/or applications 512c, such as website(s) 512d, a rules engine 512e, and/or a document manager 512f may be stored on memory 511. The website program 512d may generate websites that provide an interface to facilitate access to documents stored in a memory and/or to facilitate creation of new documents by users. The website may be accessed through the Internet, in some implementations. The rules engine 512e may apply one or more rules, such as rules provided by governmental agencies (e.g., life insurance settlement regulations, tax laws, etc.) and/or industry standards, to the documents. The document manager 512f may retrieve, allow modification of, and/or create documents. The document manager 512f may generate and/or identify signature indicators, which are associated with signature blocks (e.g., positions in documents for signature, such as near a signature line). The document manager 512f may generate signature indicators by identifying positions for signature in the documents (e.g., signature blocks) and/or identifying specified text (e.g., text in white or “hidden” text that is identifiable by the system but not visible when presented to users, text such as “signature”, “policy owner”, etc.). The document manager 512f may generate the documents and include signature indicators, such as metadata (e.g., text, software keys, etc.), that facilitate identification of signature blocks within generated documents. The blockchain stamp 512g may generate a blockchain digital fingerprint of any document stored on memory 511. This blockchain digital fingerprint can later be used to verify the authenticity of documents stored on memory 511. The blockchain stamp 512g may be provided by a Remote System 540 and be accessed through the Network 530. The user hierarchy manager 512h may generate connections between different users, allowing a plurality of both users at the same level, users above and users bellow. The user hierarchy manager 512h may inhibit user access to data 512i (e.g., a user can see data for life settlement cases submitted by them or any of the users bellow them but not of users above them). The memory 511 may also store data 512i, such as rules (e.g., life insurance settlement state regulations, best practices, etc.) to be applied by a rules engine 512e, documents, user information, user hierarchy networks, etc. The memory 511 may also include data 512i, such as the data used to generate life insurance settlement transaction documents, what text is “specified text” for purposes of identifying signature indicators, etc.

The data management device 510 also includes a processor 514 to execute the instructions 512a and/or save and/or retrieve data 512i. The processor 514 may execute the document manager 512f to generate documents, identify signature positions of the documents, and/or seal the documents so that future alteration of the documents may be evident and identifiable. The document manager 512f may generate signed documents based on received signatures; generate documents such that access to the documents (e.g., signed or unsigned is inhibited (e.g., encrypted files); and/or generate documents such that when documents are accessed, information related to the access (e.g., accessing party, time, date, type of access, whether data was modified, what portions of the document were presented, etc.) may be associated and/or stored with the generated documents. The data management device 510 also includes a communication interface 515 that may facilitate data transfer between the data management device 510 and the user computers 520 and/or the remote systems 540 using the network 530. For example, communication interface 515 may facilitate the retrieval of documents from remote systems 540 (e.g., databases, web servers, or other computer systems) using the network 530. The communication interface 515 may also receive electronic signatures from input devices of the user computer. The communication interface 515 may transmit life insurance settlement documents & signed documents to remote systems for storage.

A user may access websites 512d to review and/or electronically sign documents using a user computer 520. Some users may access the websites 512d using the user computer 520 to provide data that may be used to generate the life insurance settlement transaction documents. Some users may access the websites 512d using the user computer 520 to review life insurance settlement documents and provide offers. Some users may access the websites 512d using the user computer 520 to order additional life insurance settlement transaction documents. The user computer 520 may be a personal computer, laptop, personal digital assistant (PDA), smart phone, or other suitable computer, and may include input devices such as a digital signature pad, touch screen, stylus, etc.

As illustrated in FIG. 5, a user computer 520 may include a memory 521 to store data 522 and instructions 523, such as an operating system 523a and/or other applications 523b. The user computer 520 also includes a processor to execute instructions and/or to access and/or manipulate data 522. The user computer 520 may include a presentation interface to present, for example, webpages 512d and/or other interfaces provided by the data management device 510. The user computer 520 also includes a communication interface 526 to facilitate communication with other systems through the network 530. The user computer may also include one or more input devices, such as keyboards, signature pads, touch screens, light pens, and mice, to facilitate obtaining signatures from a user.

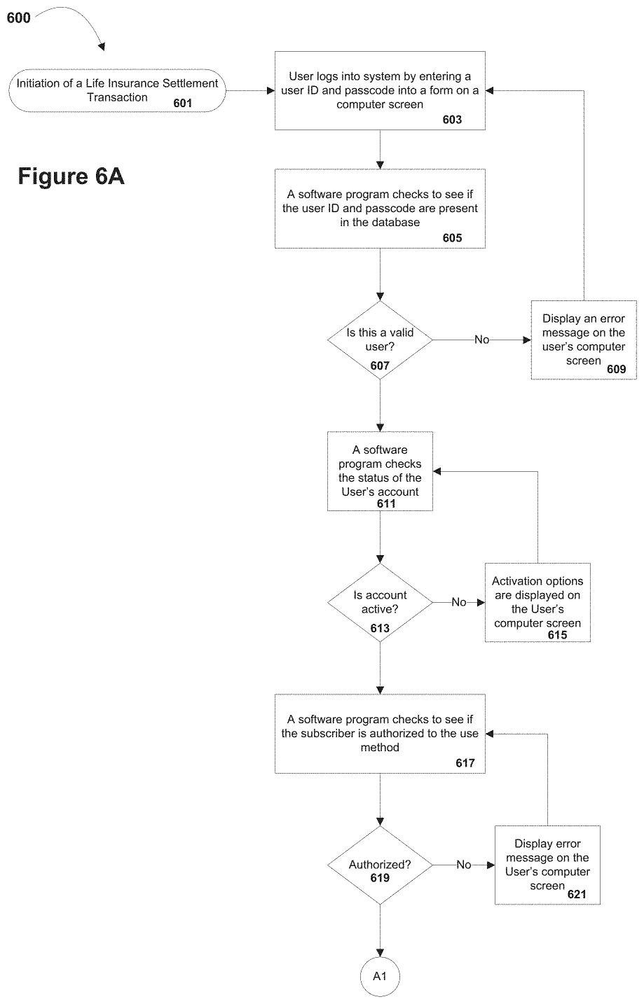

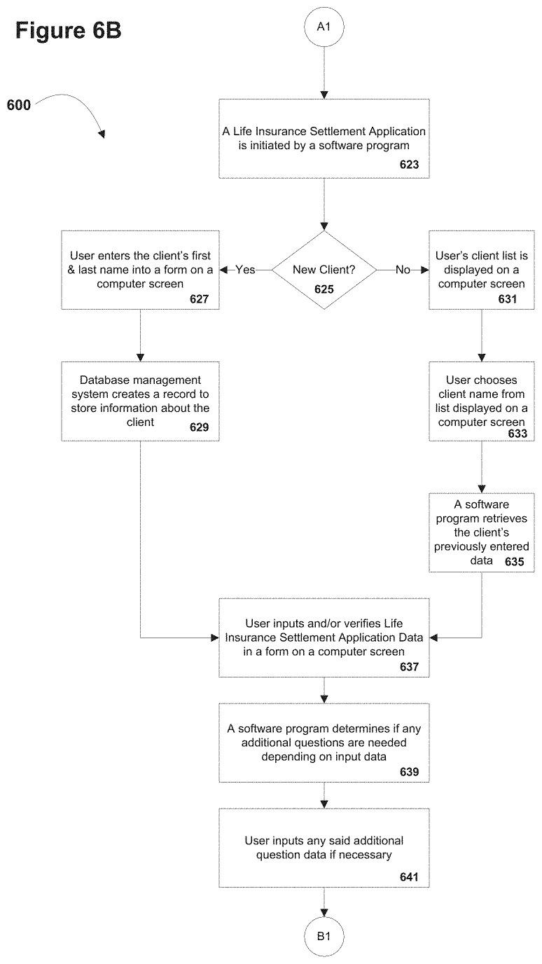

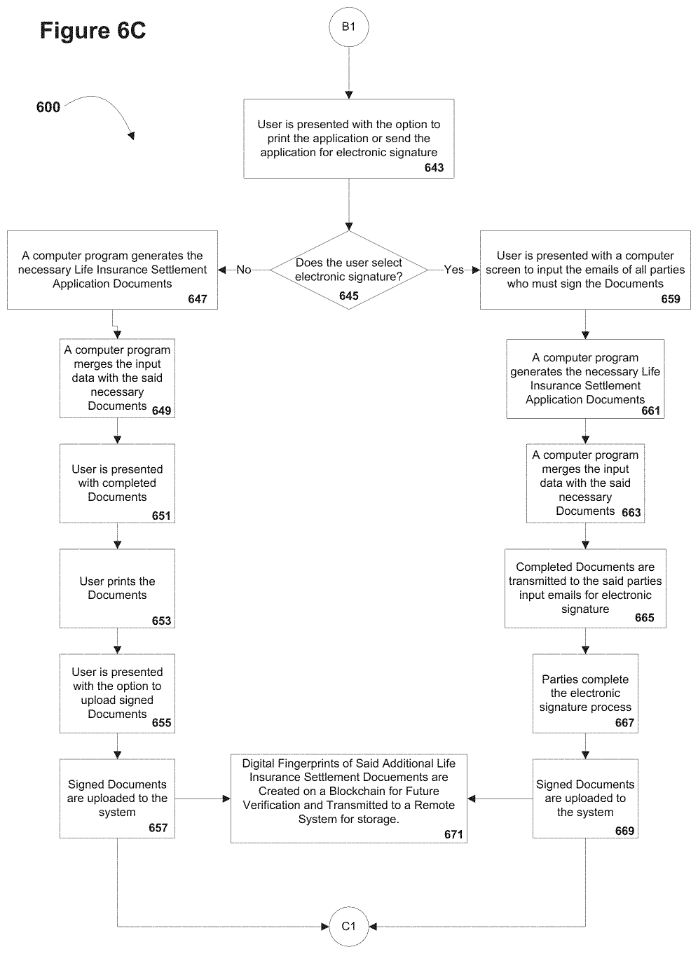

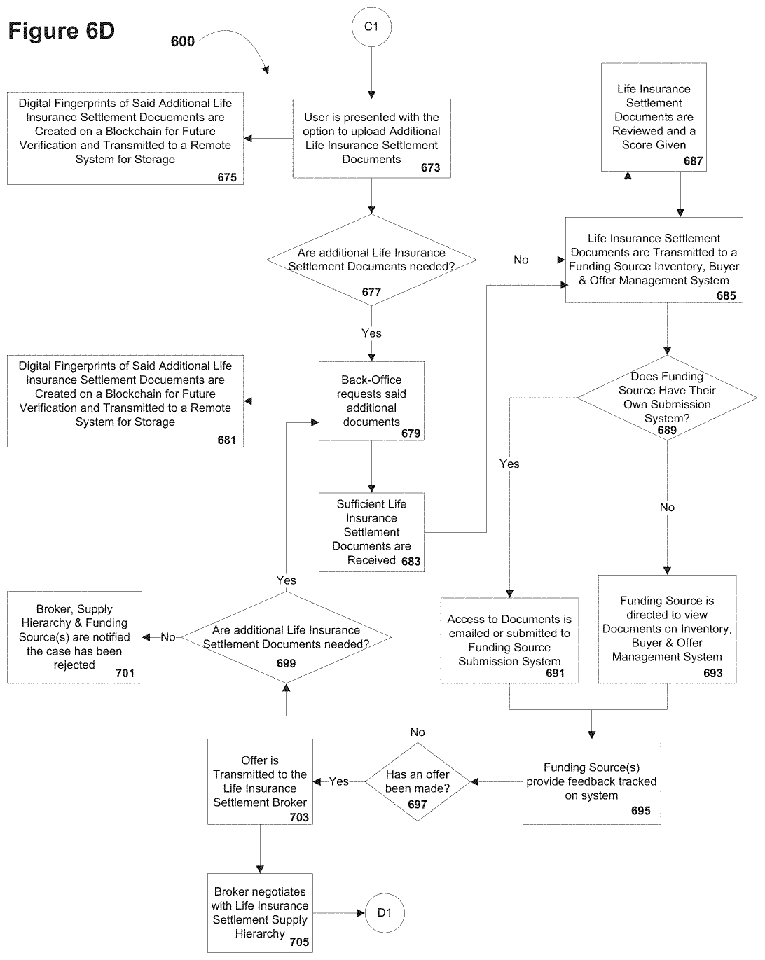

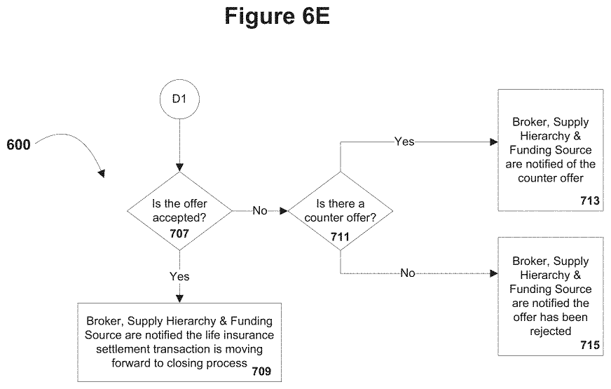

Referring to FIG. 6A-E provide a running flow diagram of one embodiment of the system and method. Once a user has registered, the user is able to initiate a life insurance settlement transaction 601 by logging on to the system and entering an identification code and pass code 603. Software of the system of the present invention provides a check on users logging onto the system for valid user ID and pass code 605, user account status 611, and authorization to use the method 617. If the user is valid with an active account and is authorized to use the method they may now use the system and method to initiate a life insurance settlement transaction 623.

Supply hierarchy users work with policy owners who want to sell their life insurance. If the client is a new client, the user enters the client's first name and last name into the system 627, and the information is stored in a new record in the system database 629. If the client is an existing client, the broker pulls the client's information from the system 635 based on the client's name 633 that is on the broker's client list 631. If a client is an existing client, some of the life insurance settlement application data may be pre-populated when being entered into the system 637. Dynamically, while the user is inputting the life insurance settlement application data a rules based software program determines if any additional data is needed 639. Once the user has input optional data and any required data to the system 641 the user is presented with the option to print the application or send the application for electronic signature 643.

If the user selects to print the application the system generates the necessary life insurance settlement application documents 647, merges the input data with said necessary documents 649, and presents the user with completed life insurance settlement application documents 651. The user can print the completed documents 653, upload the signed completed documents 655, and the signed completed documents are stored on the system 657, and a blockchain digital fingerprint of the signed completed documents is generated on one or more blockchain(s) 671.

If the user selects to send the application for electronic signature the user is presented with a computer screen to input the emails of all parties who must sign the life insurance settlement application documents 659. The system generates the necessary life insurance settlement application documents 661, merges the input data with said necessary documents 663, and completed life insurance settlement application documents are transmitted to the said parties input email addresses for electronic signature 665. Each party reviews the completed documents and electronically signs the completed documents 667. The electronically signed completed documents are stored on the system 669, and a blockchain digital fingerprint of the signed completed documents is generated on one or more blockchain(s) 671.

The life insurance settlement parties are presented with an option to upload additional life insurance settlement documents to the system 673 (e.g., copy of the policy, life insurance illustrations, medical records, etc.). If additional life insurance settlement documents are needed the signed life insurance settlement application documents may be sent to a back-office to request said additional documents 679. Once sufficient life insurance settlement documents are received 683 the life insurance settlement documents are transmitted to a funding source inventory & offer management system 685. Life insurance settlement documents may be reviewed and a score as determined by rules based criteria may be given 687.

The life insurance settlement documents are made available to a plurality of funding sources. If a funding source has their own submission system access to the life insurance settlement documents are submitted to the funding source submission system 691. If a funding source does not have their own submission system the funding source is directed to view the life insurance settlement documents on the inventory & offer management system 693. Each funding source has an opportunity to provide feedback on the life insurance settlement case 695 (e.g., offer, reject, offer pending, review pending, etc.). A funding source may not make an offer but may request additional life insurance settlement documents to be able to consider making an offer 699. These requests are transmitted to the back-office to request said additional documents 679. If no funding source makes an offer and no additional life insurance settlement documents are needed, the life insurance settlement parties are notified the case has been rejected at this time 701.

If an offer is made, the offer is transmitted to the life insurance settlement supply hierarchy top level 703. The bid- revise-bid method from system 300 or similar offer negotiation systems may be implemented to negotiate the offer to the life insurance policy owner 705. If the offer is countered by the life insurance policy owner or supply hierarchy the funding source is notified of the counter offer 713 and has an opportunity to revise their offer to reach an acceptance 709. If the life insurance policy owner rejects the offer and provides no counter offer the funding source is notified the offer has been rejected 715 and has an opportunity to revise their offer to reach an acceptance 709. If an offer is accepted, all life insurance settlement parties are notified the offer has been accepted and the transaction is moving forward to the closing process 709.

Alternatively, a user may request that a third party, also a user to the present system and method, initiate the method at the life insurance settlement stage 623.

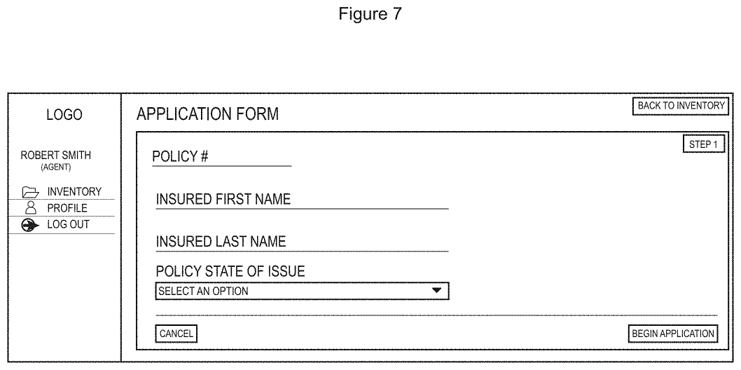

FIG. 7 illustrates an example of a life insurance settlement application form screen. Pre-application life insurance settlement data is entered on this form. The system will run the Policy State of Issue through a rules based engine to determine if any additional questions or documents will need to be completed to satisfy state regulation. Once this data is input and the “Begin Application” button is clicked, subsequent application form screens are presented requesting all the data necessary to complete a life insurance settlement application.

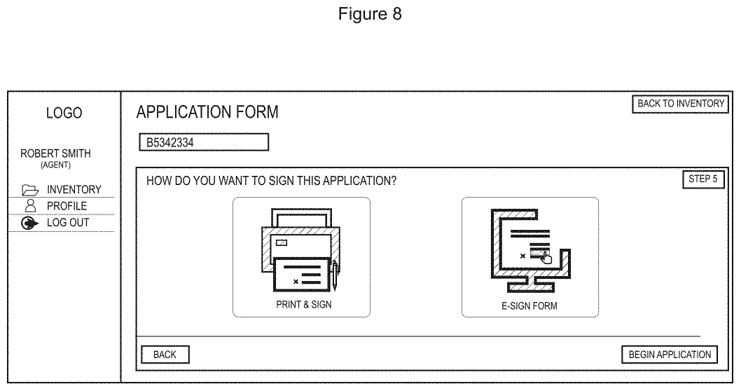

FIG. 8 illustrates an example of a life insurance settlement document signature options screen. Once all life insurance settlement application data is entered through application form screen(s) these options are presented. If E-Sign Form is selected the user is presented with a screen to input the signing parties email addresses who will receive a message with instructions on how to electronically sign the document. If Print & Sign is selected the user is presented with a completed application to print and subsequently provided an option to upload a digitized copy of the signed life insurance settlement application.

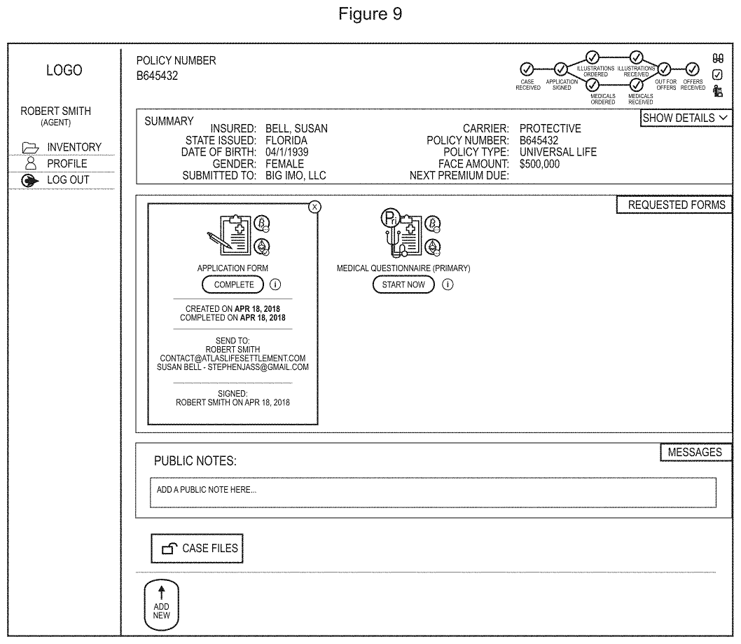

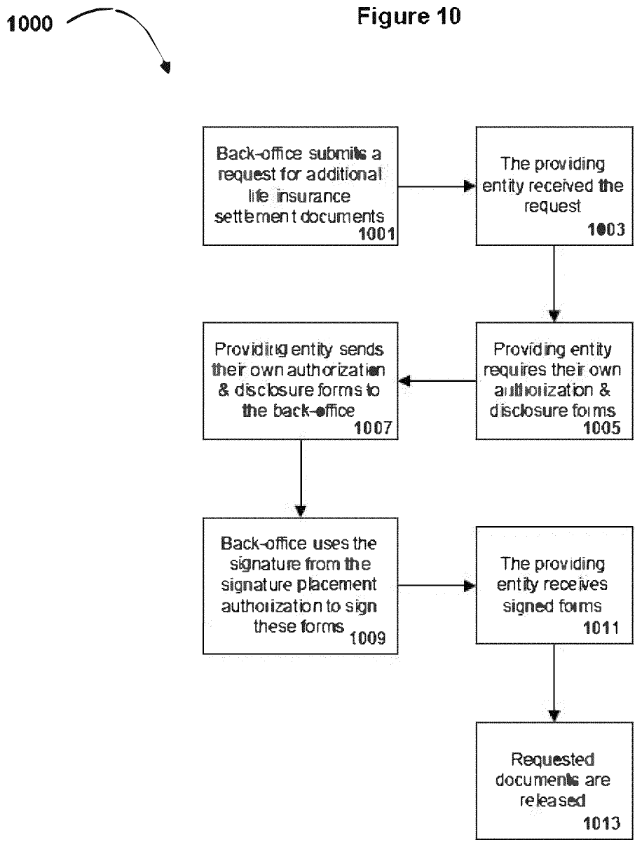

FIG. 9 illustrates an example of a life insurance settlement status screen. Once a life insurance settlement application form is complete the status timeline at the top of the screen (right) becomes green with a checkmark for “Case Received”. Once the life insurance settlement application documents are signed the status “Application Signed” becomes green with a checkmark. A back-office user can then use the signed life insurance settlement application documents to request additional life insurance settlement documents and provide order request and requirement received status to other users who have access to the life insurance settlement status screen. Once the back-office user has sent the life insurance settlement documents Out for Offers the status of funding sources interest and offers is shown. The status of the digital fingerprint generation for blockchains can be seen to the right of the Requested Forms icons. Additional requested forms can be started and completed through the Requested Forms section. Alternative embodiments of this life insurance settlement status screen will include further status systems (e.g., display of any offers that have been made, provide a way to send offers, show the status of each individual additional life settlement document request, etc.).

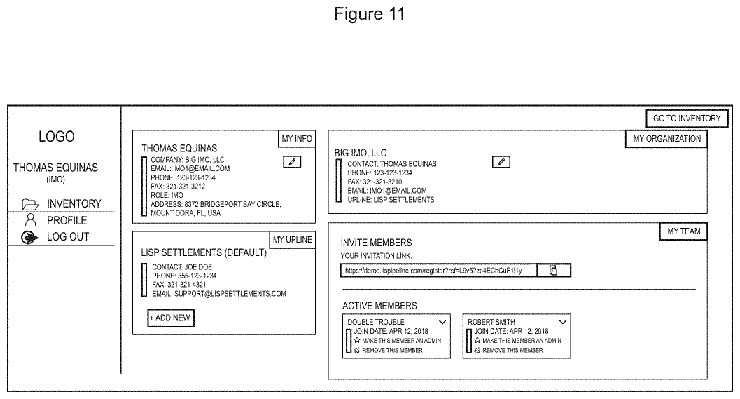

FIG. 11 illustrates an example of a user hierarchy network. In this example My Info represents a user. The My Upline represents the next level up the user hierarchy network from the My Info user. The My Organization represents an organization the My Info user may create to connect users bellow them. My Team users can be selected as Admins for the My Info user and be brought up to the same level as the My Info user. Otherwise, the My Team represents users bellow who are the next level down from the My Info user. Both My Upline and My Team users can have a plurality of My Upline and My Team users that will create their user hierarchy network. For example, a case submitted by Robert Smith would be seen by Robert Smith, Big IMO, LLC & LISP Settlements. An offer made on this case would be submitted to LISP Settlements, revised and submitted to Big IMO, LLC, revised and submitted to Robert Smith, and revised and submitted back to LISP Settlements to present to the policy owner. Additional embodiments of this user hierarchy network could include flat commission rates as well as fully transparent offers to some or all parties. Furthermore, other life settlement parties may create user hierarchy networks (e.g., funding sources, providers, etc.).

What is claimed is:

1. A system and method for preparing, processing and managing life insurance settlement transaction documents, the method comprising:
receiving a request to access life insurance settlement documents;
generating a terminal interface for the entering of information regarding a life insurance settlement transaction by a user;
merging said information with life insurance settlement documents;
generating an interface for presentation of merged life insurance settlement documents;
identifying one or more signature blocks in the presented portion of the merged life insurance settlement documents by identifying one or more signature indicators in the life insurance settlement documents, wherein a signature indicator is associated with a signature block;
requesting signature of the presented portion of the merged life insurance settlement documents; and
receiving a signature of the presented portion of the merged life insurance settlement documents, wherein the received signature is associated with at least one of the signature blocks.

2. The method of claim 1 wherein the signature indicator includes metadata that identifies a portion of the life insurance settlement application document as a signature block.

3. The method of claim 1 wherein the signature indicator may be text.

4. The method of claim 1 further comprising merging the life insurance settlement documents coupled with a print means for printing said merged life insurance settlement documents.

5. The method of claim 1 further comprising generating signed life insurance settlement documents based on the received signature.

6. The method of claim 1 further comprising generating life insurance documents by identifying specified text in the life insurance settlement transaction information. (text may be “state” or “is owner divorced” or “life insurance carrier” or “medical facility”).

7. The method of claim 1 further comprising transmitting the merged and signed life insurance settlement documents to a remote system for storage.

8. The method of claim 1 wherein the merged and/or signed life insurance settlement documents are generated such that when the life insurance settlement documents are accessed, information related to the access is stored in association with the life insurance settlement documents.

9. The method of claim 1 further comprising transmitting the signed life insurance settlement documents to a device of the party that provided the signature of the presented portion of the life insurance settlement documents.

10. The method of claim 1 wherein not all life insurance settlement transaction information has been provided prior to generating said merged life insurance documents.

11. The method of claim 1 wherein the life insurance settlement transaction information is entered through a form.

12. The method of claim 1 further comprising inhibiting signature by a notary public prior to receiving the signature of the presented portion of the life insurance settlement documents.

13. A system and method for preparing, processing and managing life insurance settlement transactions for a plurality of remotely connected users, said system comprising:
Processing means, including a data management device into which data is written and from which data is read, said data management device storing information regarding user hierarchy networks, case details, offer details, case files, and an existing life insurance policy owner(s), insured(s), beneficiary(ies), policy values, and the full text of all life settlement document provisions;
Terminal means for interactively communicating online with said processing means and accessible by a user to produce requests and to enter information and/or retrieve information for writing into and/or reading from said data management device;
Display means for displaying information that is entered and retrieved;
A data transfer server;
Merging means included in said processing means for reading out from said data management device selected policy/client information and merging said read out policy/client information to complete final life insurance settlement documents tailored to each particular policy, completed documents being provided to said data transfer server;
Print means coupled to said merging means for printing said final life insurance settlement documents; and
A forms database, said forms database including life insurance settlement forms.
Electronic signature means for allowing specified users to electronically sign life insurance settlement documents.

14. A system as in claim 15 wherein said remotely connected users are connected over remotely located terminals, said remotely located terminals being connected to said remote server over a network, users providing policy information to said forms from corresponding ones of said remotely connected terminals.

15. A system as in claim 15 further comprising signed life insurance settlement documents based on the received signature and said signed documents being provided to said data transfer server.

16. A system as in claim 15 further comprising transmitting the signed life insurance settlement documents to a back- office with requirement ordering means to gather medical records, life insurance illustrations, life expectancy reports, pricing reports, medical summaries and prescription reports.

17. A system as in claim 15 further comprising of users being able to transmit an offer amount to a policy owner.

18. A system as in claim 15 wherein offer data may be shared between a plurality of users and access to said offer data inhibited by other users.

19. A system as in claim 15 further comprising funding source users being able to make an offer comprised of one or both cash and retained death benefit amounts on a life insurance settlement case.

20. A system as in claim 15 wherein when offers are made on a life insurance settlement case, information related to the offer made is stored in association with the life insurance settlement case.

21. A system as in claim 15 wherein users can connect with other users above, bellow or on the same level to create a user hierarchy network.

22. A system as in claim 15 wherein users can connect with other users using unique registration links.

23. A system as in claim 15 wherein users can connect with other users through said terminal means.

24. A system as in claim 15 wherein users can view life settlement cases submitted by users within their user hierarchy network.

25. A system as in claim 15 wherein a status, related to the documents and transaction, is stored in association with the life insurance settlement case.

26. A system as in claim 15 wherein life insurance settlement case submissions can be sold as leads.

27. A system as in claim 1 and claim 15 further comprising of life insurance carrier forms being merged, signed and used to make life insurance carrier requests.

28. The method of claim 1 and claim 15 in which at least some of the data correspond to information that is obtained from a party other than the user (e.g., policy insured(s), policy owner(s), policy beneficiary(ies), a fraud-prevention agency, a government agency, partner website, etc.).
